(19) United States
(12) Patent Application Publication
(10) Pub. No.: US 2015/0359653 A1
(43) Pub. Date: Dec. 17, 2015

(54) HOLDING MEANS FOR COUPLING AT LEAST ONE GUIDING/SUPPORTING STRUCTURE TO A LIVING BEING

(71) Applicant: EXOS GMBH, Munich (DE)

(72) Inventors: Laura DIEZ, Kempten (DE); Greta STEHLING, Thansau (DE); Volker JUNIOR, Graefelfing (DE)

(73) Assignee: EXOS GMBH, Munich (DE)

(21) Appl. No.: 14/765,157

(22) PCT Filed: Jan. 30, 2014
(86) PCT No.: PCT/EP2014/051823

§ 371 Date: Jul. 31, 2015

(30) Foreign Application Priority Data
Jan. 31, 2013 (DE) 10 2013 001 732.7

(51) Int. Cl. A61F 5/01

(52) U.S. Cl. CPC A61F 5/0123

(57) ABSTRACT

The invention relates to a holder means for connecting, more particularly for the defined fastening, of at least one guide-support structure to a living entity. The holder means comprises at least a holder unit and a fastening means for the locally stable arrangement of the holder unit on the living entity, wherein the holder unit is elongated in design and can be arranged in the direction of extension of a body surface line of the living entity to at least in sections overlap the body surface line, wherein the body surface line is such that between two different body load conditions it essentially experiences no change in length.

In accordance with the subject matter of claim 1, the present invention relates to a holder means for connecting at least one guide-support structure to a living entity and in accordance with claim 9 to a multiple layer arrangement of a holder means and a guide-support structure.

The connection of guide-support structures, more particularly ortheses, to living entities, in particular humans, requires many different considerations to be taken into account. In contrast to the application of technical elements to, for example, mechanical devices or buildings, the bodies of living entities are always different from each other. This results, for example, from different body sizes, different muscle and tissue structures, different length ratios of the legs to the upper body and much more. As can be easily seen from this, in contrast to the connection of a guide-support structure to a mechanical device, the result is highly complex.

A guide-support structure designed as an orthesis, for example, should not slip on the connected area of the body as this is often perceived as very troublesome and the functional capacity is thereby usually restricted or impaired. In addition, it must be taken into account that living entities feel pain and the contact points, at which an orthesis is applied or can be applied, can become sore. In the case of the ortheses available on the market, they are connected to the human via the production of a strong gripping force between the orthesis and the human, through which the aforementioned drawbacks can occur.

It is therefore the aim of the invention to provide a possibility of connecting a guide-support structure to be applied to the body of a living entity in such a way that a locally stable and comfortable, connection is achieved, which, in particular, does not restrict the freedom of movement of the living entity.

In accordance with the subject matter of claim 1, the aforesaid task is achieved by a holder means for connecting, more particular for the defined fastening, of at least one guide-support structure to a living entity. The holder means comprises at least one holder unit and one fastening means for the locally stable arrangement of the holder unit on the living entity. The design of the fastening means and the type of fastening it brings about can, for example, be dependent on the number of holder units. In the case of a one holder unit or more holder units, more particularly 2, 3, 4, 5, 6, 7, 8, 9, 10, the fastening means is preferably understood as being a gripping or adhering means, by way of which all the holder units can be adhered to the living entity. The holder unit is preferably essentially longitudinal and designed in such a way that it extends in a configuration coupled with the living entity in the direction of a body surface line, more particularly a defined body surface line, of the living entity and covers this body surface line in the direction of its extent at least in sections, wherein the body surface line is such that between two different body load conditions it essential undergoes no expansion or compression, particularly preferably no change in length or no noticeable change in length.

Such body surface lines are known as “lines of non-extension” (LONEs) and describe lines along the human body that undergo no change in length (extension or compression) during movement of the body. Such lines, which experience no change in length during the movement, can be identified for all regions, parts, points of the body and limbs (or also for the entire body). A holder unit then preferably encompasses the body wholly or partially without thereby restricting the freedom of movement of the living entity. In addition, the use of such a holder unit brings about a minimisation of the relative movements of the guide-support structure to be connected and the body surface of the living entity. By means of the holder unit arranged over a LONE or in the area of a LONE, an ergonomic transmission of forces and movements from the human body to the externally applied element/connected guide-support structure or vice-versa is thus made possible in an advantageous manner.

The holder means thus constitutes a fastening system or a connection plane to fasten the most varied of guide-support structures, such as, for example, one or more protection device(s), more particular protectors, orthesis/ortheses, prosthesis/prostheses, rucksacks, shoes, lifting system(s), bag(s), exoskeleton(s), crutches or similar element(s) to the human body.

The present invention is advantageous as it provides a holder means that with its holder unit(s) is in contact with, or overlaps, body surface parts which do not undergo a change in length during a movement and are therefore locally stable. This local stability is exploited in accordance with the invention in order to connect one or more guide-support structures to the living entity in a locally stable manner. In this way, the production of forces required for traction can be reduced enormously compared with bindings which have no holder means in accordance with the invention, as no or essentially no compensation of relative movements of the guide-support structure resulting during a movement has to take place on the skin. Overall this results in a very locally stable and comfortable possibility of arranging a guide-support structure on a living entity.

The invention is also advantageous in that compared with a fully encompassing non-elastic holder means the freedom of movement of the living entity is not restricted, as the non-elastic holder units only extend along the body lines that do not change in length,

In accordance with a preferred form of embodiment of the present invention, several holder units are provided and each of these holder units can be arranged in the direction of the extension of a body line of the living entity to at least partially overlap the relevant body surface line. Preferably the holder units can be distributed over the entire body or also only on certain regions and/or points or parts of the body for use in sections. It is therefore conceivable that as large a number of holder units as possible, or a task-specific number or selection of holder units or only a few or particular holder units are used. It is also conceivable that the holder units are, or can be, individually designed for a particular person or according to assembled sizes. Advantageous here is, for example, that in accordance with a topology derived from series measurements, in a very time-saving manner and relatively inexpensively, holder means can be produced, which though not individually adapted to every living entity, nevertheless approximately optimally fit defined groups, e.g. men, skiers, leg lengths between 100 cm and 110 cm.

The body surface lines are thus characterised in that between two different physical load conditions they essentially experience no change or no change in length, more particularly expansion or compression.

This form of embodiment is advantageous as through the use/deployment of several holder units several body surface lines (several LONEs) can be overlapped or covered which can bring about even greater stability and connection security, particularly resistance to torsion, between the guide-support structure and the living entity.

In accordance with a further preferred form of embodiment of the present invention, the holder units are arranged in such a way that the totality of the holder units forms a net-like or grid-like structure. Preferably the individual holder units are connected with other holder units at individual or all contact points with other holder units or at crossing points with other holder units with the contacting further holder unit.

This form of embodiment is advantageous as the holder units together form a main structure/the net-like or grid-like structure that allows the force transmission of the forces introduced into the individual holder units into other holder units. In a usage configuration the main structure can be in direct or indirect contact with the body surface of the living entity, i.e. so that the holder units can be arranged directly on the skin surface.

It is also conceivable that the holder means can be clearly positioned due to the shape of the main structure. The shape of the main structure is preferably such that the main structure can only be applied to one defined position/setting/point on the living entity. It is also conceivable that positioning aids, such as markings etc., can be provided so that the holder means comes to rest in the correct position on the body.

In accordance with a further preferred form of embodiment of the present invention, an elastic holder medium is provided, wherein the holder units are connected to the holder medium or are an integral part of the holder medium. The holder units or some of the holder units can thus be arranged, for example, underneath or above the holder medium. In addition, the holder units, or individual holder units can be surrounded by an upper or lower layer of the holder medium. Preferably the holder units are detachably or non-detachably applied to the holder medium. Non-detachably applied holder unit are preferably sewn on, welded on, stuck on and/or stapled on. Detachably applied holder units are preferably strapped on, fixed by buttons, by means of magnets and/or tied on. The holder units are therefore preferably connected to the holder medium in a frictional, positive-fit, bonded and/or tractional manner. It is also conceivable for holder units or individual holder units to be a partially or fully integrated part of the holder medium.

In accordance with a further preferred form of embodiment of the present invention, the fastening means is formed by at least two interacting holder units and/or by the holder medium.

This form of embodiment is advantageous as on the one hand, due to its shape the main structure formed by several holder units allows fastening of the holder means to the living entity. On the other hand, or additionally, it is conceivable that the holder medium, if it is essentially tubular in design, for instance, due to its elastic properties produces a pressing on effect onto the relevant part of the body through which if preferably forms a frictional connection with the body part surface and also reduced the overall displacement of the skin over the remaining body and/or positively influences muscle functions.

In accordance with a further preferred form of embodiment, the holder medium is designed as an item of clothing. The holder medium can preferably be considered as a holder layer which is preferably formed by close-fitting clothing, such as compression clothing. It is also conceivable for the holder medium to comprise several layers which are made of the same or of different materials. The item of clothing formed by the holder medium can be designed for the lower body, for example, more particularly in the form of trousers or a stocking, for the upper body, more particularly in the form of a pullover, long-sleeved shirt or a T-shirt or for the upper body and the lower body, more particularly in the form of overalls.

This form of embodiment is advantageous as the holder means can work independently of a guide-support structure and can in certain applications act as temperature and/or light and/or vision protection. It is also conceivable for the holder medium to be designed to remove and/or draw off moisture produced by the body part. It is equally conceivable for the holder means to preferably be arranged partially under and/or over the guide-support structure to be attached, or for it to partially or completely enclose, surround or include it.

In accordance with a further preferred form of embodiment of the present invention the holder unit for connecting the guide-support structure overlaps the holder medium on the side facing the guide-support structure and at least one further holder unit is arranged on the side of the holder medium facing the living creature and overlaps the holder medium.

This form of embodiment is advantageous as it allows highly stable connection to the living entity, and the holder medium between the layers of holder material can preferably form an elastic or partially elastic intermediate component.

In accordance with a further preferred form of embodiment of the present invention the holder mean has at least one connecting element or precisely one connecting element or several, more particularly 2, 3, 4, 5 and 6 connecting element for connecting the guide-support structure, wherein the at least one connecting element is arranged on the outside of the holder means and, overlapping or adjacent to the holder means and the holder medium and/or at least one holder unit in sections. In addition or alternatively to the connecting element, a connection area can be envisaged/formed on the holder means to connect the guide-support structure to the holder means.

The connection/fixing/coupling of single or multiple holder units, more particularly the thereby preferred grid-like or net-like main structure, or the holder means to the guide-support structure preferably takes place in a non-detachable manner and particularly preferably in a detachable manner, more particularly in a reversible or repeatable manner. Preferably the connection between the holder means/the main structure and the guide-support structure can produced in a frictional, positive, bonded and/or tractional manner. Examples of techniques which can be used on their own or in combination with one or more further techniques include, for example, sticking, sewing, riveting, screwing, strapping, impregnating/infiltrating, integrated production of laminating. Alternatively or additionally it is conceivable that the carrier means can be connected to the body and then via one or more holder structure with the desired element of the desired guide-support structure.

In accordance with a further preferred form of embodiment of the present invention the surface of at least one holder unit is at least in sections designed to form a frictional and/or positive connection with the guide-support structure.

This form of embodiment is advantageous as the guide-support structure can only be fastened in sections or completely without belts or straps to the holder means.

In accordance with a further preferred form of embodiment of the present invention at least one holder unit is essentially or completely non-elastic. Seen as non-elastic or essentially non-elastic are holder units which have an E-module of ≧0.5, preferably ≧1 and preferably of ≧5 and particularly preferably of ≧10 or 15 or 50 or 100 or 200. Individual parts of the holder units or the entire holder units consist, for example, of non-elastic textiles or almost non-elastic textiles, which are preferably in a strip, fibre or band-like form or which are processed, more particular woven and/or chemically treated. It is also conceivable for plastic materials, solid adhesive or rubbers, more particularly strip-like or band-like, to be used. Additionally or alternatively it is also conceivable that one, some or all holder units comprise(s) bands of natural fibres or combinations thereof or have/has such bands or fibres.

This form of embodiment is advantageous as the forces introduced into a holder unit can at least partially be directed directly into one or more holder units whereby a distribution of the introduced force onto different body surface areas at a distance from each other is brought about.

In addition, a multiple layer arrangement in accordance with a further preferred form of embodiment of the present invention has a holder means for connecting, more particularly defined fastening, of at least one guide-support structure to a living entity comprising at least one holder unit and one fastening means for the locally stable application of the holder unit on the living entity, wherein the holder unit is longitudinal and can be arranged in the direction of extension of a body surface line of the living entity to at least overlap the body surface line in sections, wherein the body surface line is such that it essentially undergoes no extension between two different body stress conditions. The multiple layer arrangement also comprises a guide-support structure, more particularly an orthesis, for the transmission of forces that can be introduced into a first section of the guide-support structure to a second section of the guide-support structure at a distance from the first section, wherein the first section is designed for at least direct and at least sectional face-to-face contact on the surface area of the living entity's body. Essential is that at least the first section of the guide-support structure at least in parts overlaps the holder unit or at least one holder unit.

This form of embodiment is advantageous in that through the overlapping of the holder unit(s) by the guide-support structure of the guide-support structure one or more body points optimally suited for stable application on a living entity is/are predetermined.

In accordance with a further preferred form of embodiment of the present invention, at least the planar section of the first section for application on the body part is designed in such a way that in an alignment vis-a-vis the body part which is envisaged for fulfilment of the function of the guide-support structure it can be applied on surface areas which on transition from a first load condition into a second load condition exhibit surface deformations which during the same transition are less than surface deformations of further surface structure of the body part, or it can be applied in a defined alignment envisaged for the functioning of the guide-support structure on surface areas which on transition from a first load condition into the second load condition exhibit surface deformation which during the same transition are greater than the surface deformations of other surface area of the body part.

This form of embodiment is highly advantageous as the guide-support structure is designed in such a way that it causes the living entity no constriction and is thus highly comfortable to wear. The invention is also advantageous as compared with a fully encompassing non-elastic holder means it does not restrict the living entity's freedom of movement as the non-elastic holder units only extend along the body lines that experience no change in length. In addition, in connection with the holder means it is ensured that the guide-support structure does not slip out of place, which prevents the risk of incorrect functioning.

Preferred as living entities within the framework of the invention are humans, more particularly, children, adults, seniors and/or physically disabled persons and/or injured persons and/or sportsmen/women, and/or animal, more particularly household pets such as dogs and/or cats, or farm animals such as horses, cows and/or pigs.

The planar section preferably comprises several, more particularly exactly two, two or more than two, more particularly exactly three, three or more than three, more particularly four, four or more than four areas with which the guide-support structure can be connected to the body part or body parts, particularly preferably in a positive-fit manner.

In addition, the first section is preferably not only designed for contacting the surface of a body part indirectly and in sections, but can also be in direct contact with the body, wherein it is conceivable that the first section is not only in face-to-face contact with the surface of the body part in sections, but is essentially in full or full face-to-face contact with the body part. The first section of the guide-support structure and/or the holder unit is preferably of a solid material or a combination of several materials, more particular carbon, PE, PP, PA etc. and composite materials such as carbon fibre composite material or glass fibre composition material, which preferably provides the guide-support structure and/or the holder unit strength. However, preferably this material combination is not in contact with the body part or the surface of the body part in the state when connected to the body part. The first section of the guide-support structure preferably also has a soft material or a combination of materials, more particularly foam, textiles, rubber etc., which are softer than the solid materials and preferably can be brought into contact with the surface of the body part at least indirectly and preferably directly.

Compared with the ortheses known from the state of the art, a guide-support structure which is preferably designed as an orthesis has significant advantages. Among other things, the guide-support structure in accordance with the invention can be used in particular for injury prevention in sports, as on the basis of the adaptation to the relevant body part(s) in accordance with the invention, in comparison with ortheses known from the state of the art, no, or considerably fewer or negligible constrictions of the body part(s) occur, more particularly in the connection area, i.e. between the guide-support structure and the body surface, more particularly in the area of the holder means during different stress states, more particularly muscle tension states. The guide-support structure in accordance with the invention thus preferably serves to ergonomically take up forces or transmit forces, moments and/or movements from the body of a living entity, more particularly from a human body, to the guide-support structure applied on the body or a holder means and/or the other way round. The guide-support structure in accordance with the invention and the holder means in accordance with the invention as well as a combination thereof can thus be used considerably more comfortably than the known ortheses for the prevention of injury during increased stress condition, for example in recreational sports or even in performance sports, or in the prevention of injury, more particularly in the case of long, i.e. several-hour or long-term working tasks or as part of an exoskeleton in the domain of rehabilitation facilities, more particularly for the defined alignment of two or more body parts in relation to each other or for the alignment of two or more parts of a body part, e.g. when splinting a broken arm.

In addition or alternatively to connection by means of the holder means, the guide-support structure can also be fixed by means of belts, more particularly elastic/flexible belts and or lacing, more particularly elastic/flexible lacing, to the relevant body part(s) and/or to clothing. In the event that the guide-support structure is integrated into clothing, the guide-support structure is preferably non-detachably or detachably connected with the clothing, preferably the guide-support structure can be or is velcroed to the clothing. The clothing can, for example, be close-fitting elastic clothing and/or a holder means in accordance with the invention.

The planar portions, more particularly the soft components, of the guide-support structure that can be directly or indirectly brought into contact with the surface of the body part are preferably applied to the other components, more particularly the solid components, preferably in a positive-fit, bonded, frictional and/or tractional manner, e.g. glued on, sewn on, riveted on, welded on, pressed on, screwed on, integrated, laminated and/or combinations thereof. The planar sections(s) of the guide-support structure for coming into contact with the body parts can also be designated as body-adapted surfaces, which preferably, can be formed or produced in order be brought into contact with any kind of body regions, parts, points and limbs.

In accordance with a further preferred form of embodiment of the present invention, the planar section is designed so that it can predominantly or exclusively or essentially completely be brought into contact with the surface areas of the holder means or the body part, wherein during the transition from a first load condition into a second load condition the surface regions of the body part deform less that the further surface regions of the body part.

The surfaces of the guide-support structure therefore advantageously surround the body or the body part without restricting the freedom of movement or change in muscle volume or surface deformation of the body part, more particularly not in an unintentional manner and also minimise the relative movement between the guide-support structure and the body or the body part(s).

In a further preferred form of embodiment, the second section of the guide-support structure is designed in such a way that it can be applied to a further body part, more particularly a lower limb, that it can be applied to the holder means and/or the surface areas of the further body part in the alignment with regard to the body part, more particularly an upper limb, defined for fulfilling the function of the guide-support structure which on transition from a first stressing state into a second stressing state exhibit surface deformations that are smaller compared with surface deformations of other surface regions of the further body part during the same transition, or to surface areas of the further body part which on transition from a first stressing state into a second stressing state exhibit surface deformations which are greater than surface deformation of further surface regions of the further body part during the same transition.

This form of embodiment is advantageous as the guide-support structure, more particularly as an orthesis, can be comfortably attached and/or used on several parts of a body part and/or on several parts of the body of a living entity, more particularly a human.

In accordance with a further preferred form of embodiment of the present invention the shape of the planar section can be determined in accordance with a process described below or the guide-support structure is produced in accordance one of the processes described below.

The determination or creation of the shape of the planar section in accordance with the method according to the invention ensures that the guide-support structure necessary or required for the living entity or area of use in question, preferably optimally, i.e. entirely individually, fits the living entity and thereby highly advantageously reduces the risk of constrictions and/or any possibly incorrect stressing.

In accordance with a further preferred form of embodiment of the present invention, the shape of the planar section is determined through local surface deformation data which is ascribable to groups of persons and/or groups of body parts and kept in a data collection and can be determined on the basis of the following method(s).

This form of embodiment is advantageous as, for example, on the basis of a topology or surface structure or surface characteristic or surface form or surface deformation distribution derived from series measurements, in a very time-saving and thus comparatively cost-effective manner guide-support structures can be designed and/or produced, which though not adjusted to every living entity individually, are nevertheless an approximately optimal fit for defined groups, e.g. men, skiers, leg lengths between 100 cm and 110 cm.

According to a further preferred form of embodiment of the present invention, the planar section forms two partial sections, which can at least partly surround the surface area of the body part and the holder means and between which there is a recess, wherein a surface area assignable to the recess on transition from a first stressing state in to a second stressing state exhibits greater surface deformations that those of the surface areas that can be overlapped by the planar section and/or the recess is designed so that in a tensed state the muscles of a living entity extend into the recess or between the planar sections or between the planar section further than is the case in an relaxed state, or surface area assignable to the recess on transition from a first load condition into a second load condition exhibits smaller surface deformation that those of the surface areas overlapped by the planar section.

This form of embodiment is advantageous as through the at least partial surrounding of a body part and the holder means by the guide-support structure, the latter can be connected to the body part or to the carrier means in a positive-fit manner. The planar sections are therefore particularly preferably designed so that when correctly used they are in contact with areas of smallest volume change of the body part or smallest contact form changes of the surface of the body part during a particular movement or activity. In addition, the recess forms an area which permits surface area deformations of the body part without constrictions taking place.

In accordance with a further preferred form of embodiment, the two partial sections of the planar portion are connected to each other via a component extending in the area of the recess, more particularly an at least sectionally elastic and/or flexible component. Preferably the component has a tendency to be deformable. The component can for example be a band, a net, a textile, a membrane and/or a combination thereof. The use of an elastic component is advantageous as the overall stability of the guide-support structure is increased, the component, due to its elastic properties, does not, or only unessentially, constricts the body part of the living entity and/or by means of the elastic component slipping off of the guide-support structure is prevented further or reduced or inhibited.

In addition it is conceivable that a part of a further preferred form of embodiment, the guide-support structure is an integral part of the holder means, the holder medium or an item of clothing, more particularly trousers, a T-shirt, a pullover, a jacket etc.

The present invention can also relate to a method of determining the surface characteristics of body surfaces. This method comprises the stages of aligning at least one body part of the living entity in relation to a recording device for the at least partial recording of the surface deformations of a least one surface region of the body part, recording initial surface characteristics or shapes of the surface region by means of the recording device, wherein the body part is in a first load condition, more particularly a state of relaxed muscles and/or in a state with body parts aligned in a neutral position with regard to each other, the recording of second surface characteristics or form of the surface region by means of the recording device, wherein the body part is in a second load condition, which is different from the first load condition, more particularly a state of tensed muscles and/or in state with body parts inclined and/or rotated and/or pivoted towards each other, and determination of the local surface region deformations resulting from between the first load condition and the second load condition, more particularly by mean of an evaluation of the recorded surface characteristics or forms or through a comparison of the recorded surface form, more particularly through a comparison of the first surface form with the second surface form.

Preferably a guide support structure can among other things be specifically understood as an orthesis, more particularly an arm, knee, joint, leg, hip, shoulder, trunk, neck, nape, hand or foot orthesis, a prosthesis, an exoskeleton, a holder system, more particularly a rucksack, a bag, a climbing belt, a lifting system, a rucksack, a walking aid, more particularly a crutch, a shoe, more particularly a ski shoe, a protection devices, e.g. a bandage, especially for use in sports, or a protector, more particularly for the prevention of accidents at work and/or sports injuries, a stabilising device, such as a splint for splinting bone fractures etc. and/or as a combination thereof. In all these forms of embodiment the holder means can be, or is, at least in sections arranged between the body surface of the living entity and the guide support structure.

Preferably one, or particularly preferably more, identical or different devices can be designated as the recording device. Particularly preferably the recording of surface deformations or varying body part volumes takes place by way of sensor devices, that are attachable to the body part or the surface regions and are designed, in particular, for pressure recording, and/or an optical recording device, more particularly an image recording device or a video recording device, and/or by means of an emitter device for the emission of rays that penetrate the body part, and/or a mechanical and/or fluidic recording device and/or a terahertz scanner, wherein the sensor devices can be applied around the body part, more particularly are arranged on a hose-like means and wherein the image recording device is preferably a 3-D scanner and wherein the emitter device is preferably an X-day device, a CT scanner or an MRI scanner.

The alignment of a body part in relation to a recording device is preferably to be understood as the introduction of the body part into the operating range of the recording device and/or the bringing of the body part into a specific position in relation to the recording device.

At least sectional recording of surface deformation preferably describes the fact that not all surface deformations have to be recorded, wherein particularly preferably all surface deformations of a body part are recorded.

The term surface characteristics preferably describes the surface structure or the topography of the surface, more particularly the skin and/or the coat of the living entity. Particularly preferably this should be understood macroscopically, i.e. characteristics or structure or form recorded from deformations, more particularly extensions and contractions of, for example, more than 1 mm and preferably more than 3 mm and particularly preferably more than 5 mm.

This method is advantageous as through the determination of the local surface deformation surface regions can be identified which, in spite of normal muscle activity and even during sporting activity experience considerably fewer or no deformations in comparison with other surface regions of the same body part. Guide-support structures can be advantageously attached to these slightly deformed or undeformed surface regions as the risk of constrictions is greatly decreased due to the considerable reduction in the expansion. The guide-support structures can therefore be attached with considerably less force to the body part in question. Furthermore, through the attachment of a guide-support structure, more particularly in combination with a holder means in accordance with the invention, slipping of the guide-support structure on the slightly deformed or undeformed surface regions is practically ruled out, as a result of which the freedom of movement is advantageously retained and/or the intended or defined or envisaged movement restriction is ensured.

Preferably the method is thus used for the determination of surface regions of a body part of a living entity that are suitable for bringing into contact with guide-support structures for the unidirectional or bidirectional transmission of forces and/or moments, wherein the surface regions are preferably characterised in that in comparison with other surface regions of the same body part exhibit smaller deformations between two load conditions or for the determination of surface regions that behave statically or essentially statically between different load conditions, or for the determination of at which position of the recorded surface region which degree of deformation in relation to an initial state occurs.

The method can preferably be used or adapted for supporting the production of a guide-support structure and particularly preferably for a production method for the production of guide-support structure.

In accordance with a preferred form of embodiment of the present invention a comparison of the recorded local surface deformations with an at least defined threshold value or a defined threshold value function is carried out, wherein deformation values resulting therefrom, more particularly below the defined threshold value or the defined threshold value function, are describable, definable and determinable as values of low surface deformation, more particularly less expansion and contraction and, inversely, more particularly above the defined threshold and/or the defined threshold value function as describable, definable and determinable as values of higher surface deformation.

Here it is conceivable that not only a threshold value and/or a threshold value function is envisaged for the identification of areas of lesser and greater deformation, but that multiple-stage, more particularly three-, four, five-stage, more particularly more than five-stage, different grades of dynamics/statics are identifiable. Thus, for example, all areas whose deformations are below a first threshold value can be designated as static, all areas whose deformations are above the first threshold value but below a second threshold value, can be designated as partially dynamic, and all areas whose deformations are above the second threshold value can be designated as dynamic. These different areas can, for example, be specially marked optically, more particularly they can be coloured in accordance with a colour scheme assigned to the relevant degree of deformation.

This form of embodiment is advantageous as through the threshold value(s) and/or the threshold value function(s) the determined results can be optically processed for and thus issued in an easily understandable manner to a person carrying out or supervising the process or for documentation purposes. Furthermore, it is conceivable that in an automated manner using the optimum connection areas determined by means of the threshold values and/or threshold value functions, a processor unit can generate a view, more particularly a 3-D view of the body part and/or guide-support unit, and/or a production plan, and/or initiate or implement or directly derive the production of a guide-support structure.

In accordance with another preferred form of embodiment the determination of the local surface determinations and/or the comparison of the recorded surface deformations is brought about by a processor device. The processor device can be, for example, a component part of the recordering device and/or be connected to the recording device.

Preferably the determination and/or the comparison by the process device take place fully automatically or partially automatically.

The processor device preferably comprises a storage device for storing raw data and/or processed data, wherein it is also conceivable that the storage device and the processor device are only connected to each other by signals, i.e. for the exchange of data. Additionally or alternatively the processor unit is preferably connected to an output device, such as a screen, a printer, an optical drive, and directly controllable production device, more particularly a 3-D printer etc. Preferably the processor device is equipped with processing means or has access to such processing means, more particularly a computer-aided design tool, by means of which the deformation values can be converted into data or file formats suitable for the design or construction of a guide-support structure.

In accordance with a further preferred form of embodiment, depending on at least two defined threshold values or threshold value functions at least three surface regions are assigned to zones of different surface deformation, more particularly the surface deformations in the first zone assignable to the first surface region are less that the surface deformations in the second zone assignable to the second surface region and less than the surface deformations in the third zone assignable to the third surface region, whereby the surface deformations in the third zone take place in relation to a centre of the body part preferably opposite to the surface deformations in the second zone. However, it is also conceivable that the surface deformations in the first zone assignable to the first surface region are greater than the surface deformations in the second zone assignable to the second surface region and smaller than the surface deformations in the third zone assignable to the third surface region.

In accordance with a further preferred form of embodiment of the present invention a view corresponding to the deformation values and/or to surface regions assignable to the one side of the threshold value or the threshold value function is created and/or shown to illustrate different degrees of deformation at different positions of the surface region, whereby particularly preferably the deformation values and/or the surface regions assigned to one side of the threshold value or the threshold value function are converted into data for the production of a 3-D model, more particularly of a body part and/or a guide-support structure adapted to the body part.

This form of embodiment is advantageous as the determined results can be issued optically processed and therefore in an easily understandable manner to a person carrying out or supervising the process and/or for documentation purposes. In addition, by way of optical processing verification of the outputted deformation values can be directly verified, more particularly by a person operating the recording device.

The present invention can also relate to a computer program product that comprises a program code which is stored on a computer-readable medium in order to carry out at least individual processing stages, which have been previously described or are described below, and are necessary for recording the deformation values and/or their processing when the computer program product is run on a computer, or relates to a computer-implemented method in accordance with the previously described method. However, it is also conceivable that the present invention relates to a control means or control software, more particularly for controlling the recording device and/or the processor device and/or a production device.

It is also conceivable that the invention relates to a method of producing guide-support structures connectable to a living entity, wherein recording surface deformations of body surfaces of living entities takes place to determine suitable contact surface for guide-support structure to be brought into contact with the surface of the living entity, or that the invention can relate to a method of producing a device for fastening different element on the human body.

The production process or the method preferably comprises the stages of aligning at least one body part of the living entity in relation to a recording device, more particularly a 3-D scanner, for the at least partial recording of surface deformations of at least one surface region body part, of recording first surface characteristics or form or the surface region by means of the recording device, wherein the body part is in a first load condition, more particularly a state with relaxed muscles, or recording second surface characteristics or for of the surface region by means of the recording device, wherein the body part is in a second load condition, different from the first load condition, more particularly a state of tensed muscles, wherein during the recording in the different load conditions it is conceivable that external influences are taken into account and/or recording is carried out with but also without contact with any devices, and of determining the local surface deformations resulting between the first load condition and the second load condition, wherein, in particular, a 3-D comparison of the body surface of the different scans is carried out.

In further stages, additionally or alternatively the surfaces or positional displacements suitable for optimum fitting of a guide-support structure to a living entity are preferably extracted and, particularly preferably, adapted to specific marginal conditions or tasks, such a load scenarios. In the case of an orthesis it is conceivable that forces can or have to be absorbed in order to restrict, prevent or influence for example, valgus or internal rotation movements, for which the connection surface or the planar section of the first portion must be appropriately adapted in terms of design and/or production technology.

Preferably production control takes place based on the 3-D or CAD data, wherein it is also conceivable that the data can be used as a template for non-industrial production. The 3-D scans of the application-specific body regions cannot only be recorded in different body positions or body posture or body part positions, more particularly with different load conditions, but can also be recorded during a movement or extracted from a recorded sequence of movements. Preferably at least two or precisely two scans are carried out, namely an initial and an end scan of the movement amplitude, wherein preferably one further or further, more precisely exactly two or more than two, more particularly precisely three or more than three, more particularly four or more than four scans are carried out between the initial and end position.

Depending on the function of the guide-support structure, in accordance with an additional or alternative processing stage, a reference system is defined or determined. Particularly preferably, in a further stage a 3-D comparison of the surface deformations or an indirect or direct volume comparison of the body parts in the various load conditions is carried out, wherein preferably a definition of the local displacement take place. This can take place using the reference system or with reference thereto.

Production of such a guide-support structure preferably takes place by way of a generative process and/or using additive production, whereby in the production process preferably laminating tools and/or tools for sewing, knitting, weaving are used. It is also conceivable that in an additional or alternative processing stage, more particularly a production process stage a tool is produced which preferably remains in the subsequent guide-support structure. Preferably a production process is used in which the 3-D data can directly, or in processed form, serve as the basis for production control. It is also conceivable that connection elements are envisaged as further built-on components for connection with the body of the living entity, more particularly the human body. These connection elements are preferably for connecting or tying or fixing the guide-support structure to the body part(s) and/or for connecting two further elements can be attached to the living entity via the guide-support structure. These connecting elements are preferably designed a belts, more particularly as Velcro belts, flexible Velcro belts, leather belts, textile belts, woven belts or as lacing elements. The connection elements are consciously arranged or can be arranged in the rear areas of the guide-support structure so that preferably on the rear of the body part or on the side opposite the connection means force can be applied for the frictional and/or positive-fit fixing of the guide-support structure on the body or on the body part.

Further advantages, aims and properties of the present invention will be explained in the following description of the attached drawing, in which, holder means and a guide-support structure in accordance with the invention as well as combinations thereof are shown by way of example. The components of the holder means and guide-support structures in accordance with the invention which in the drawing at least essentially correspond with regard to their function, can be denoted with same reference number, wherein these components do not necessarily have to be given a number or explained in all the figures.

Individual or all illustrations in the following figures are preferably to be seen as design drawings, i.e. the dimensions, proportions, functional relationship and/or arrangements preferably correspond precisely or preferably essentially to those of the device or the product in accordance with the invention.

### In these

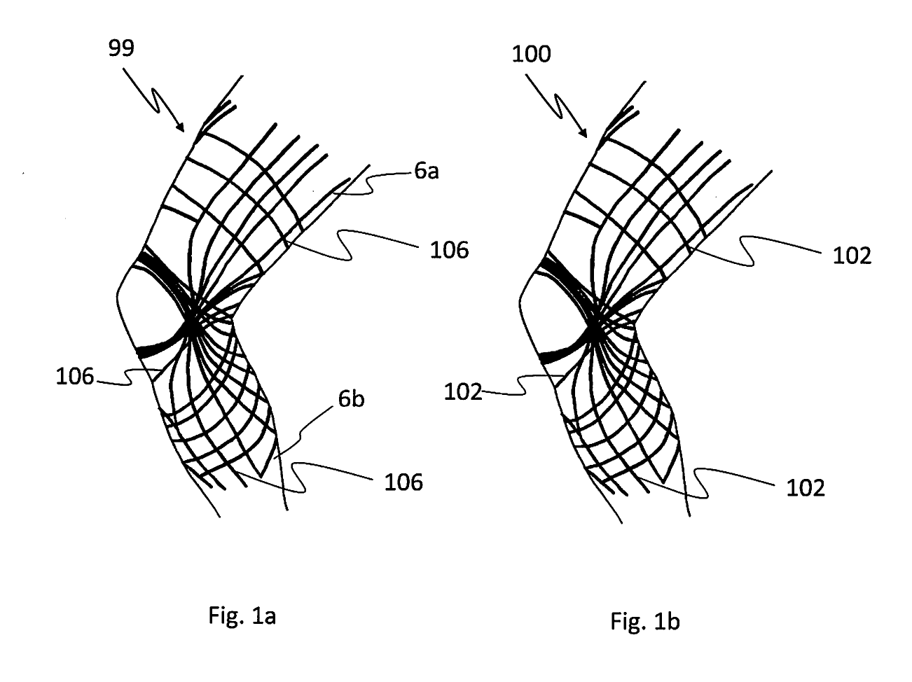

FIG. 1a shows a side view of a human leg with body surface line marked on it which do not changes in length on movement of the leg;

FIG. 1b shows a side view of a holder means in accordance with the invention applied to a leg of a human;

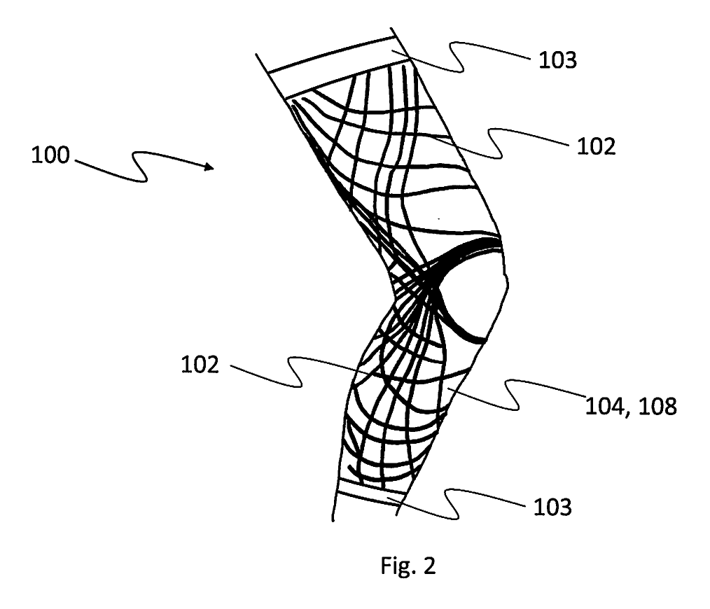

FIG. 2 shows a further side view of a further form of embodiment of a holder means in accordance with the invention applied to a leg of a human;

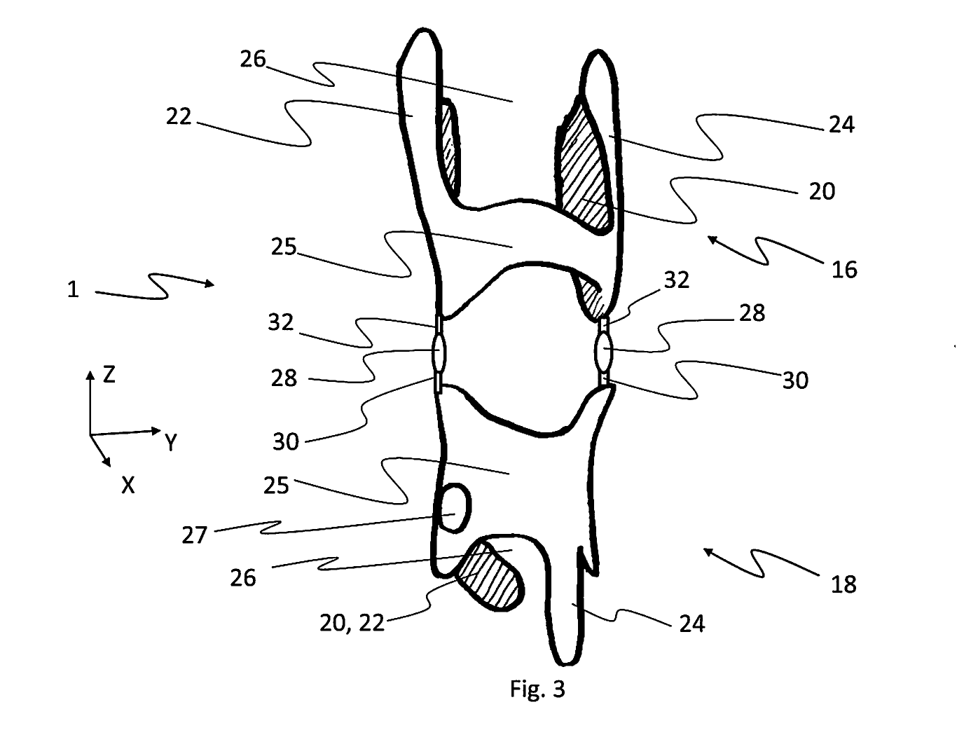

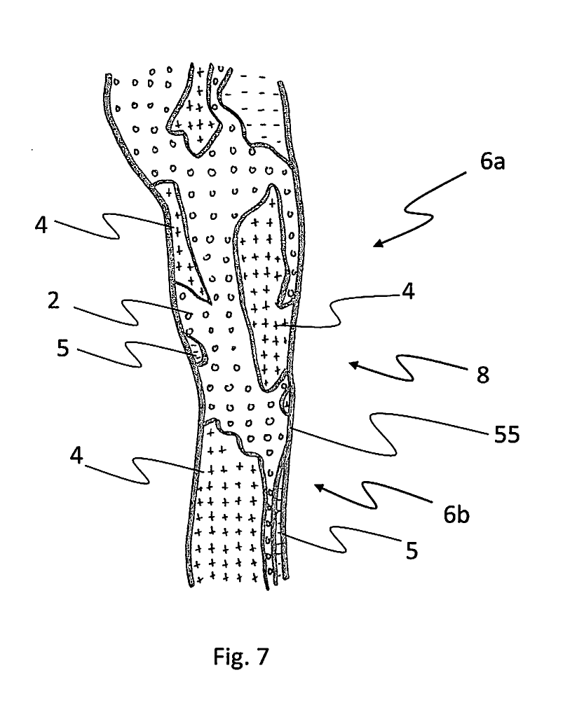

FIG. 3 shows a schematic view of a guide-support structure, which is designed taking into consideration the areas of different surface deformation shown in FIG. 7;

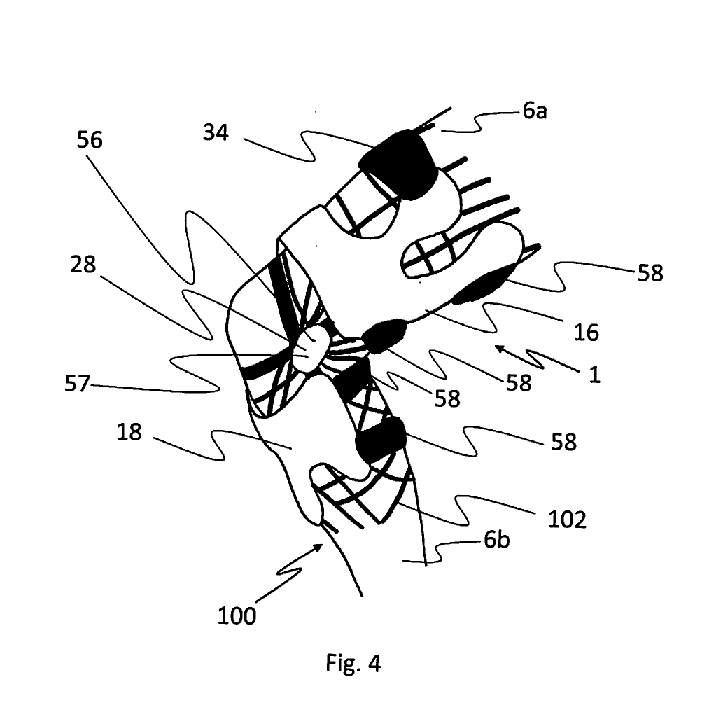

FIG. 4 shows a combined arrangement of a holder means in accordance with the invention and a guide-support structure designed as an orthesis;

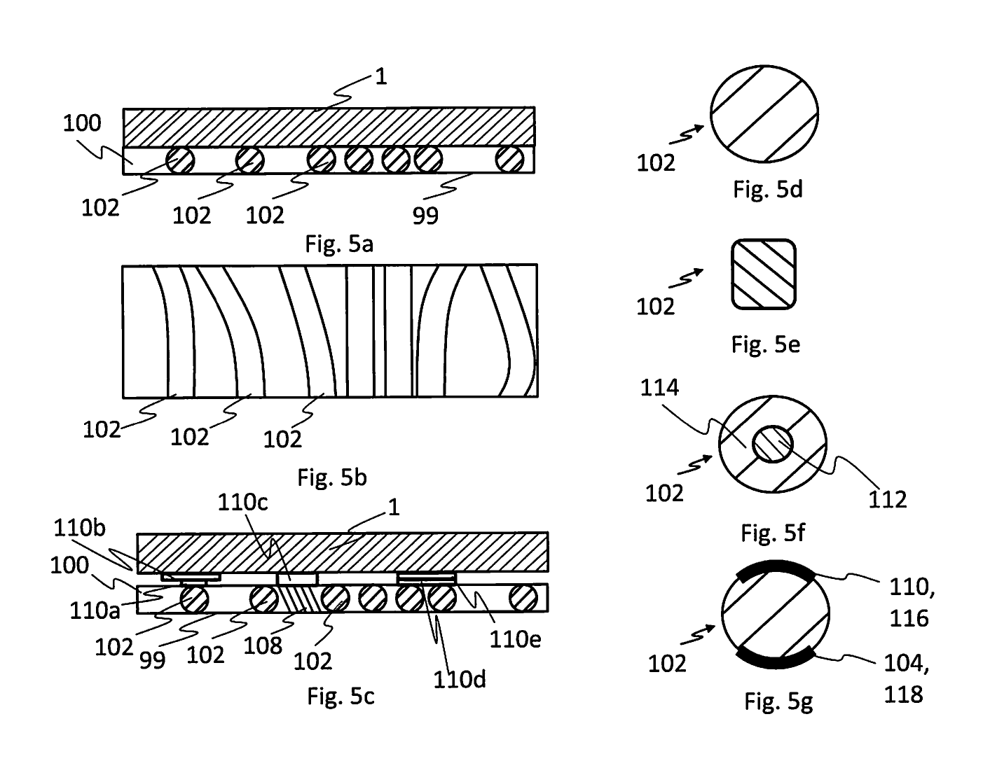

FIG. 5a shows a side view of a multiple-layer arrangement in a first cross-section, wherein the arrangement comprises a holder means and a guide-support structure in accordance with the invention;

FIG. 5b shows view from above of the arrangement shown in FIG. 5a, wherein the upper layer shown in FIG. 5 a is cut away;

FIG. 5c shows a further cross-sectional view, wherein from this cross-sectional view various example arrangement of the connecting elements can be seen;

FIG. 5d shows a cross-sectional view of a first embodiment of a holder unit,

FIG. 5e shows a cross-sectional view of a second embodiment of a holder unit,

FIG. 5f shows a cross-sectional view of a third embodiment of a holder unit;

FIG. 5g shows a cross-sectional view of a fourth embodiment of a holder unit;

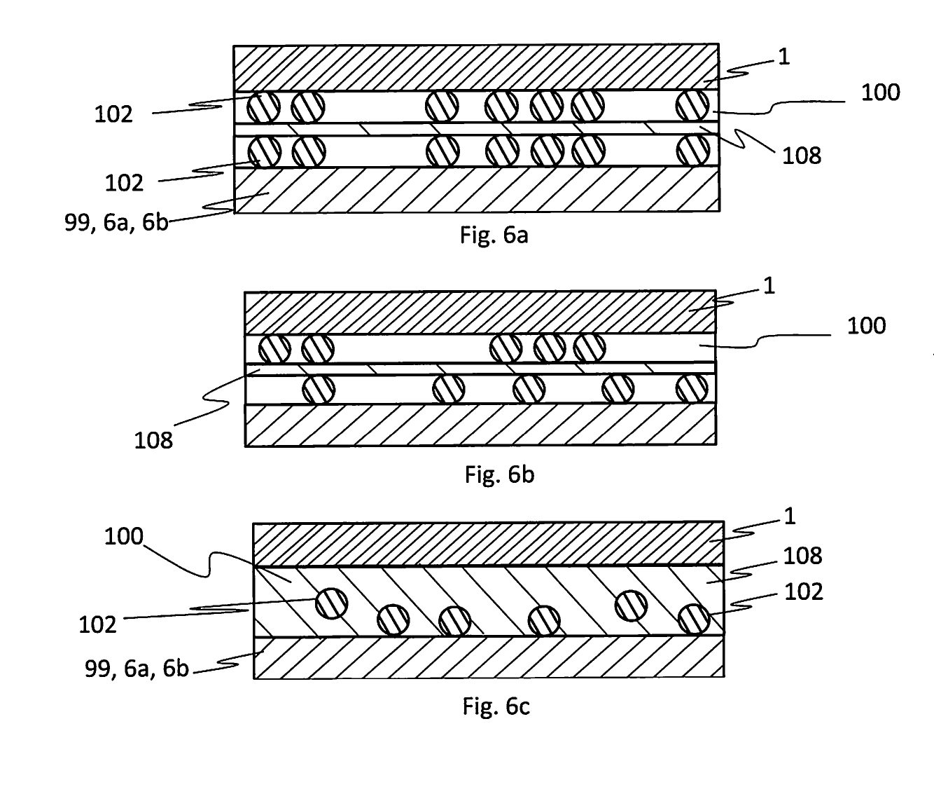

FIG. 6a shows a further side view of a multiple layer arrangement in accordance with the invention in cross-section;

FIG. 6b shows yet another side view of the multiple layer arrangement in accordance with the invention in cross-section;

FIG. 6c shows yet another side view of the multiple layer arrangement in accordance with the invention in cross-section;

FIG. 7 shows a view of areas of different surface deformation of a body part or a person determined by means of the method in accordance with the invention;

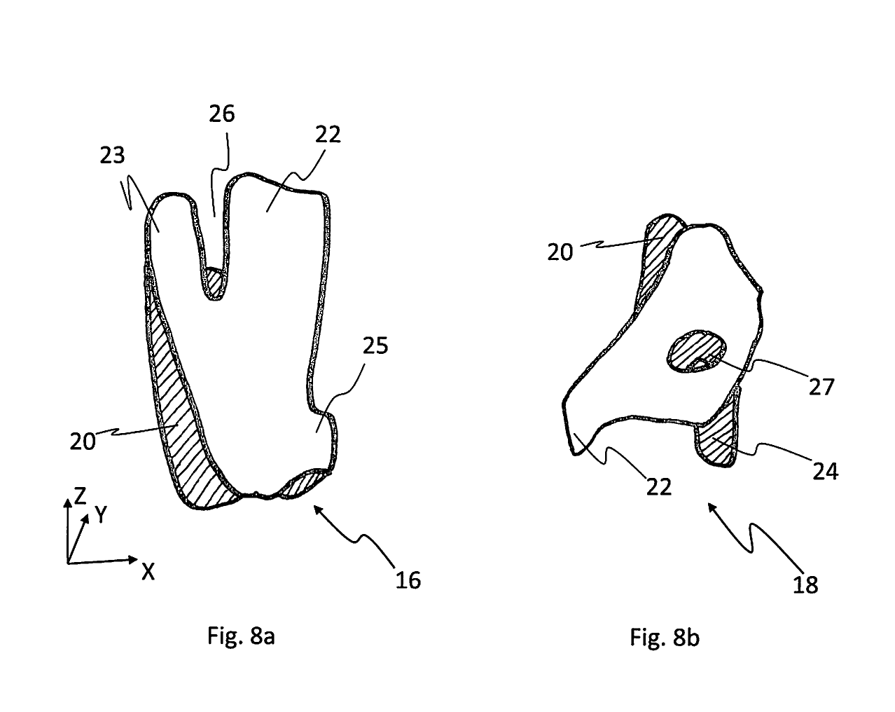

FIG. 8a shows a schematic lateral view of the upper section of the guide-support structure shown in FIG. 3;

FIG. 8b shows a schematic lateral view of the lower section of the guide-support structure shown in FIG. 3;

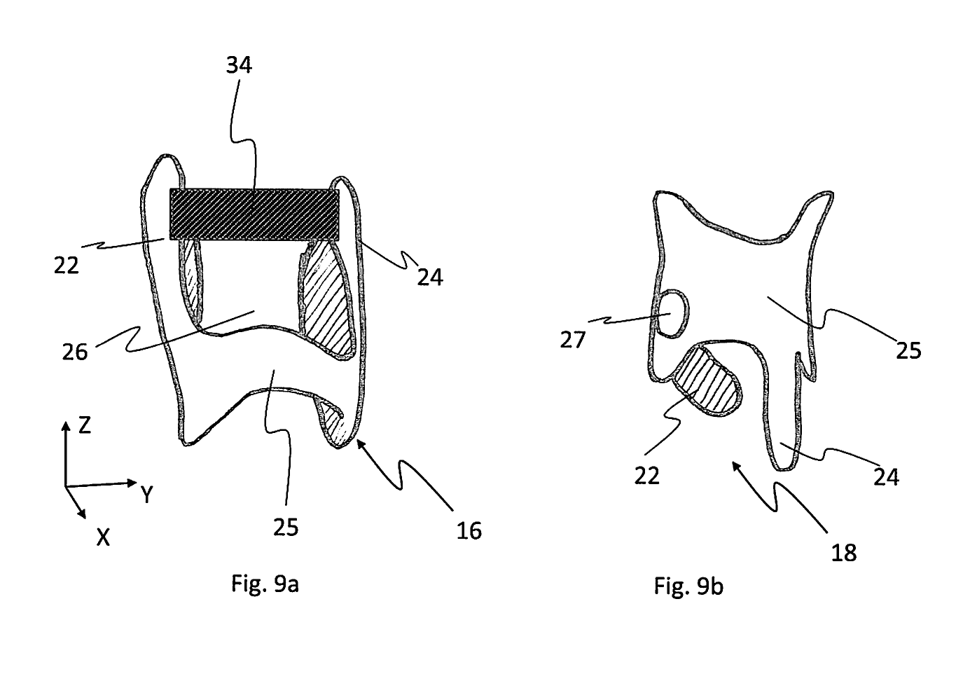

FIG. 9a shows a schematic front view of the upper section of the guide-support structures shown in FIG. 3 wherein a preferably flexible and/or elastic component is also shown;

FIG. 9b shows a schematic front view of the lower part of the guide-support structure shown in FIG. 3;

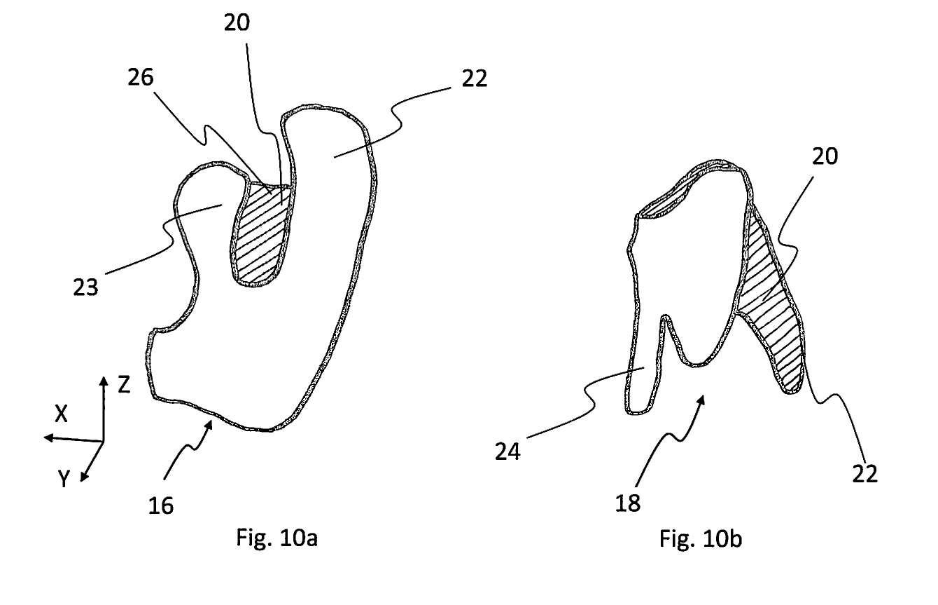

FIG. 10a shows a schematic medial view of the upper part of the guide-support structure shown in FIG. 3;

FIG. 10b shows a schematic medial view of the upper part of the guide-support structure shown in FIG. 3;

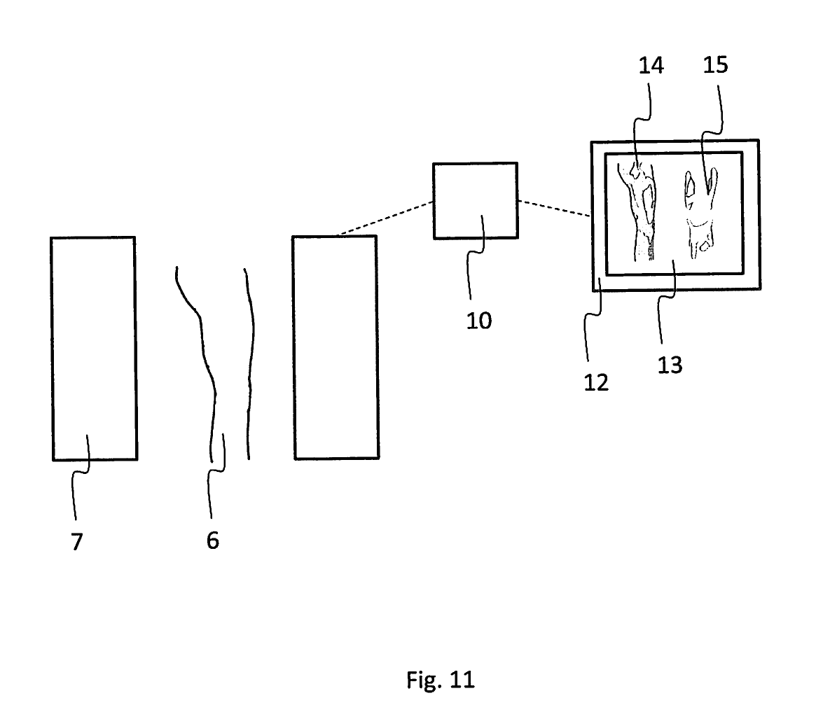

FIG. 11 show an example of a device arrangement by means of which the method in accordance with the invention can be carried out.

FIG. 1a shows a body part 99, more particularly a human leg. Shown on the upper leg 6a and the lower leg 6b are body surface lines 106 which during a movement of the leg, more particularly a movement of the lower leg 6b to the upper leg 6a experience no change in length.

In FIG. 1b a holder means 100 in accordance with the invention for connecting guide-support structures to the body of the human are shown. The holder means 100 has holder units 102 which are designed in such way that they overlap the body surface lines 106 shown in FIG. 1a. The number of holder units 102 can be set to any number or in accordance with use. For example, it is conceivable that the holder means 100 has more or fewer holder units 102 than body surface lines 106 shown in FIG. 1a. From this figure it can be seen that the carrier means 100 can be designed in a net-like or grid-like manner.

FIG. 2 shows a further form of embodiment of the present invention, according to which individual or all holder units 102 terminate in an end band 103 of the holder means 100 in the region of their lower end and/or in the region of their upper end. This embodiment is highly advantageous as it greatly facilitates the application of the holder means 100 to the body of a living entity. Through the overall or main structure that they form, the holder units 102 can bring about locally stable positioning of the holder means 100 on the body 99, as a result of which the holder units 102, particularly in their entirety, can be considered as fastening means 104. In addition, between the individual or all holder units 102 and/or the holder units 102 overlapping at the top and/or bottom, there can be a holder medium 108 which additionally or alternatively can bring about fastening of the holder means 100 to the living entity. Depending on the design, the holder medium 108 can thus also be seen as, or constitute fastening means 104.

FIG. 3 shows a schematic perspective view of a preferred guide-support structure 1, more particularly a leg or knee orthesis. The orthesis 1 has an upper section 16 and a lower section 18. The upper section 16 is preferably designed in such a way that it is attachable or can be attached to the upper leg 6a of the leg shown in FIG. 7 in the manner according to the invention. The lower section 18 of the orthesis 1 is preferably also designed in accordance with the recorded surface regions 2 and/or 4 and/or 5, i.e. in such a way that it can be attached to the lower leg 6b shown in FIG. 7 in the manner according to the invention. The upper section 16 and the lower section 18 are preferably connected to each other via one or more flexible connection pieces 28 and particularly preferably via one joint 28 or two joints 28. The joint(s) 28 can be directly arranged or formed on the upper section 16 and/or the lower section 18, wherein preferably they are arranged on the first and section 16, 18 by means of lower bindings 30 and/or upper bindings 32.

The upper section 16 preferably comprises at least one first partial section 22 and second partial section 24 for laterally or bilaterally surrounding the upper leg 6a through which the upper section 16 can be attached to the upper leg 6a, more particularly to the holder means 100 (FIG. 1b) in a positive-fit manner. The two partial sections 22, 24 preferably extend in the longitudinal direction of the leg 6 or the upper leg 6a and are preferably connected to each other by a connection means 25 preferably extending in the circumferential direction of the leg. Particularly preferably the partial sections 22, 24 and any other partial section 23 (cf. FIG. 8a) as well as the connection means 25 joint form a unit produced in one piece. However, it is also conceivable for individual elements 22, 23, 24, 25 or all elements 22, 23, 24, 25 to be detachably or replaceably connected to each other. In accordance with this figure the partial sections 22, 24 delimit a recess 26. The recess 26 is an area in which the body part 6 or the upper leg 6a can experience greater volume changes or surface deformations, more particularly expansion and contraction, in comparison with the areas overlapped by the partial areas 22, 24, without resulting in constriction of the body part 6 during a change in volume of the body part 6 or during a deformation of the body part surface 4. On the inside, at least one partial section 22, 23, 24 and preferably the first partial section and/or the second partial section 24 and, more particularly the first partial section 22, the second partial section 24 and precisely one further partial section 23 or at least one further partial section 23 are designed as a planar section 20 so that at least in sections it can be applied or arranged face-to-face and at least indirectly to the surface of the body part 6, more particularly to surface regions 2 which between two load condition experience no or essentially no changes in volume or surface deformations. It is also conceivable that the connection element 25 preferably extending in the circumferential direction of a body part 6 is also designed to be planar on the inside for application to the body surface 2 or bringing into contact with the carrier means 100 and/or the body surface 2. If the connecting means 25 is arranged or envisaged in the region of a strongly deforming body surface 4, it is preferably designed in such a way that it cannot or only insignificantly be brought into contact with the body surface 4.

With regard to the elements 20, 22, 24, 25 of the second section 18, the previously described features can be used in an analogue manner. It can also be seen from the figure that the first partial section 22 of the second section 18 extends in a different direction from the second partial section 24 of the second section 18. The longitudinal axis of the first partial section 22 preferably extends in the longitudinal direction of the guide-support structure 1 and in the circumferential direction of the guide-support structure 1. In contrast, the second partial section 24 essentially extends in the longitudinal direction Z of the guide-support structure 1 or exclusively in the longitudinal direction Z of the guide-support structure 1. Nevertheless, it is also conceivable that in a further form of embodiment the longitudinal axes of both partial sections 22, 24 extend at least partially in the circumferential direction and in parallel, or essentially in parallel to the longitudinal axis Z of the guide support structure 1.

From the figure it can also be seen that the connection means 25 of the upper section 16 extends less in the longitudinal direction of the guide-support structure 1 than the connection means 25 of the second section 18. It is however also conceivable that this relationship is inverted or that both connection means 25 extent essentially equally or precisely equally in the longitudinal direction of the guide-support structure 1. The partial sections 22 and 24 of the second section 18 preferably delimit a recess 26 which is designed for as little restriction or limitation of deformations of the body surface 4 of a living entity as possible. From this figure it can be seen that the recess 26 of the second section 18 is offset or rotated in the circumferential direction of the guide-support structure 1 in relation to the recess 26 of the first section 16. Depending on the area of application, living entity etc. it is conceivable that the recesses 26 of the first and second section 16, 18 are aligned offset or essentially flush or precisely flush with regard to each other.

It can also be seen from the figure that both the upper and also the lower section 16, 18 each form preferably two, or precisely two, or at least two recesses 26. The number of recesses 16 preferably depends on the number of partial sections 22, 23, 24, which can differ, preferably in accordance with the area of application, living entity and/or body part etc.

Reference number 27 denotes a further recess, more particularly a hole. The shape of the recess 27 can comprise straight sections and/or spherical sections, wherein it is conceivable that it only comprises straight sections or only spherical sections. The recess 27 can be designed to reduce the weight, to decrease tension and/or expose surface regions 4, more particularly to allow any type of surface deformation in the exposed or not overlapped surface region 4.

FIG. 4 shows a multiple layer arrangement wherein the first layer applied to the body part 99 is formed by the holder means 100 and the second layer by guide-support structure 1 at least overlapping the holder means 100 in sections. In accordance with this figure the guide-support structure 1 is in the form of an orthesis. Preferably in the region of its first section 16 and/or in the region of its second section 18 the guide-support structure 1 is applied to, more particularly overlaps, the holder means 100. The first section 16 of the guide-support structure 1 has an elastic component 34, which can be designed, for example, as an elastic band, more particularly a rubber band. This figure also shows that the first section 16 can also have tension means 58, which can be designed as elastic components 34 or partially elastic, fully elastic or non-elastic belt or lacing elements, more particular as velco straps. Similar, identical or equivalent tension means 58 can also be provided on the second section 18 of the guide-support structure 1. The lower section 18 is preferably connected via a joint 28 with the upper section 16. Preferably the joint 28 has an axis of rotation wherein the joint 28 preferably has more than on axis of rotation, more particularly a first and a second axis of rotation 56, 57.

This multiple-layer arrangement is advantageous as through the holder means 100 rotation of the guide-support structure 1 around the body part 99 and/or slipping in longitudinal direction of the body part can be further reduced or prevented.

FIG. 5a shows a cross-section through a multiple-layer arrangement of a holder means 100 and a guide-support structure 1. From this figure it can be seen that the individual holder units 102 are arranged partially at equal and partially at unequal distances from one another. The distance between the holder units 102 can preferably be any distance, and more particularly is definable in dependence on the determined body surface lines 106. From this figure it can also be seen that holder units 102 are preferably in or can be brought into direct contact with the body 99.

FIG. 5b shows a view from above of the arrangement shown in FIG. 5a, wherein the guide-support structure 1 is cut away. The individual holder units 102 extend in a grid-like manner and partly in different directions. Preferably every holder unit 102 is assigned to a specific body surface line 106 and therefore extends in accordance with the direction of extension of the relevant body surface line 106.

In FIG. 5c another cross-sectional view is shown. In accordance with this figure one or more connecting elements 110, more particularly connecting element pairs, are provided. The connecting elements 110a-110e cited and described below can be used individually or in combination to connect the holder means 100 to the guide-support structure 1. The connecting element 110a is arranged in the area of a holder unit 102, more particularly overlapping this holder unit 102. Preferably the connecting element 110a can be arranged directly on the holder unit, wherein it is also conceivable that it can be arranged at a distance from it. The connecting element 110a can form the same, a larger or a smaller surface than a further connecting element 110 suitable for arranging the guide-support structure 1 on the holder means with this connecting element 110a. A connection element 110 suitable for this can, for example, be the connecting element 110b. The connecting element 110b is preferably arranged on the guide-support structure 1, wherein it is also conceivable that it is or can be formed as an integral part of the guide-support structure 1.

Two interacting connecting elements 110, more particularly the individual connecting elements 110a and 110b as well as 110c and 108 as well as 110d and 110e can interact in frictional, positive-fit, bonded and/or tractional manner. Particularly preferably individual or all connecting elements interact by means of velco connections.

The connecting element 110c is preferable fastened, more particularly adhered to, welded to and/or sewn to the guide-support structure 1 and preferably interacts with a/the holder medium 108 of the holder means 100. The holder medium 108 can thus be designed on one side or both sides, in sections or fully in such a way that it can interact with the connecting element 110c for fixing the guide-support structure 1 to the holder means 100.

Furthermore, from the examples of connecting elements 110d and 110e it can be seen that these can in sections, or completely, overlap several, more particularly for example 2, 3, 4, 5, 6 holder units 102. From the example of connecting element 110e it can be seen that the connecting means on the guide-support structure side can also be applied in recesses, notches and/or recesses of the guide-support structure 1.

The properties of the aforementioned connecting elements 110a-110e can be implemented partially or fully or in a combined manner in a connecting element 110 or in a connecting element pair.

FIG. 5d shows a cross-section through an example holder unit 102 perpendicularly to the longitudinal direction of the holder unit 102. The holder unit 102 is shown as being essentially or precisely round. It is however also conceivable that a holder unit 102 in such a cross-sectional view only has spherical, more particularly curved sections in sections. It is also conceivable that a holder unit 102 is symmetrical or asymmetrical in cross-section.

FIG. 5e shows a section through a further example holder unit 102 perpendicular to the longitudinal direction of the holder unit 102. The holder unit 102 is essentially or precisely rectangular, wherein the edges are preferably rounded. It is also possible that the holder units 102 are not only used in embodiments which have round or angular cross-sections, but also in embodiments that form any manner of combinations thereof.

FIG. 5f shows an embodiment of a holder unit 102, which has a core 112 and cover 114 which surrounds the core 112 preferably partially, at least partially or completely. The core 112 can, for example, consist of a non-elastic material or a non-elastic material combination and the cover 114 can consist, for example, of a material or material combination which is more elastic than the non-elastic material. The core 112 can comprise, for example, carbon, polyester, cotton or combinations thereof and the cover 114 can comprise, for example, silicone or a silicone-like material.

FIG. 5g shows an embodiment of a holder unit 102 which is coated on the upper side and lower side 116, 118. It is conceivable that the holder unit 102 is only coated in sections or completely on both sides. It is also conceivable that the holder unit 102 only has one coating 116 or 118 in sections or completely. The coatings 116, 118 can, inter alia, be applied, more particularly sprayed-on, painted-on etc. or stuck on.

The coating 116 preferably acts as the connecting element 110, i.e. for connecting the holder unit 102 with a guide-support structure 1 or the holder medium 108. The coating 118 preferably acts as the fastening means 104 and can, for example, consist of or contain an adhesive means.

The holder units 102 shown in FIGS. 5d, e, f, g can comprise or at least partially be formed purely from textile materials, material mixtures, plastics, silicones, polymers, natural materials such as, for example, rubber and/or combinations thereof.

FIG. 6a shows an example of a multiple-layer arrangement. The multiple-layer arrangement has a body 99, 6a, 6b, a holder means 100 arranged over the body area and guide-support structure 1. The holder means 110 can consist of several layers. Preferably the holder means 100 has a holder medium 108 which on the lower side and upper side is connected to the holder units 102. The holder units 102 arranged on the upper and lower sides can be arranged essentially or precisely on top of each other and preferably orthogonally to their longitudinal direction extend less than 30 mm, particularly preferably less than 20 mm and highly preferably less than 10 mm, more particularly essentially, precisely or less than 9, 8, 7, 6, 5, 4, 3, 2 or 1 mm.

From FIG. 6b it can be seen that the holder units 102 arranged on the lower side and upper side of the holder medium 108 can also be offset with regard to each other. It is also conceivable that the holder means 100 has the same number of holder units 102 on the lower side and the upper side, wherein different numbers are also conceivable. Preferably there are more holder units 102 arranged on the lower side of the holder medium 108 than on the upper side of the holder medium 108 or vice versa.

FIG. 6c shows an embodiment variant in which in which some or all holder units 102 are partially or fully arranged in a holder medium 108. In the circumferential direction the holder units 102 can be fully or only partially be surrounded by the holder medium 108. However, preferably some or all the holder units 102 are surrounded by the holder medium in sections or partially, more particularly so that be in contact with the body part 99, 6a, 6b.

Additionally or alternatively it is also conceivable that the holder medium 108 is coated with a substance or material adhering to the body part 99 along the body surface lines 106 on the side interacting with the body 99, 6a, 6b. Preferably the coating is linear and arranged in such a way that it can overlap the body surface lines 106 (LONEs) at least in sections along their direction of extension.

FIG. 7 shows a view of a body part 6 (6a, 6b). The body part 6 is a human leg and preferably comprises at least an upper leg 6a and a lower leg 6b. The upper leg region 8 which in this view covers almost the entire upper leg 6a and more than half of the lower leg 6b of the leg 6 is divided into various zones. In this example precisely three different zone type 2, 4, 5 are shown, whereby the reference number 2 denotes a zone of small volume changes or body surface that deforms little and the reference numbers 4, 5 denote zones of great volume changes or body surfaces that deform strongly. The surface deformations in first zone 2 assignable to the first surface region are preferably smaller than the surface deformations in the second zone 4 assignable the second surface region and preferably smaller than the surface deformations in the third zone 5 assignable to the third surface region, wherein in relation to a centre of the body part the surface deformations in the third zone 5 take place in the opposite direction to the surface deformations in the second zone 4. The third zone 5 can therefore characterise a zone in which the surface deformation takes place a result of volume decrease and the second zone 4 can characterise a zone in which the surface deformations takes place as a result of an increase in volume. The first zone 2 is preferably a zone in which no or essential no surface deformation occurs.

Reference number 55 denotes a further zone. The further zone 55 preferably represents areas in which although no or only slight deformations occur, due to the solid components, such as bones or cartilage or implants which lie very closely under the surface they cannot be assessed as being suitable for connecting with a guide-support surface 1. Data relating to such zones 55 are preferably predeterminable and/or can be called up from a data memory or a data library and/or identified before surface recording on the body.

FIG. 8a shows a view of the upper section 16 in a lateral alignment. A further partial section 23 can be seen, which together with the first partial section 22 delimits a recess 26. In addition, the connection area 25 is designed in such a way that it preferably extents outwards at right angles from the centre or the longitudinal direction of the guide-support structure 1. This form of embodiment offers the advantage that the connecting means 25 overlaps the body part 6 of the living entity at a distance in such a way at it cannot be brought into contact with the body surface 4. Preferably the hatched areas are planar sections 20 which are designed to be applied to the living entity.

FIG. 8b shows a view of the lower section 16 in a lateral alignment. This view clarifies the orientation of the first partial section 22 of the lower section 18, more particularly its partial extension in the longitudinal direction and circumferential direction. Also, the preferably round or oval or essentially round or essentially oval shape of the further recess 27 can be seen. It is also conceivable that further recesses 27, more particularly holes, are or can be formed additionally or alternatively in the second section 18 and/or in the first section 16.

FIG. 9a shows a view of the upper section 16 in a frontal alignment. According to this view, the first partial section 22 and the second partial section 24 of the first section 16 are connected to each other by an elastic component 34, which can, for example, be in the form of a belt, band, woven material. The elastic component 34 can be arranged or provided on the upper section or in the area of the lower end of the guide-support structure 1, wherein it is also conceivable that additionally or alternatively it can be arranged or provided in the middle section of the recess 26 or in the lower section of the recess 26. It is also conceivable that the elastic component 34 essentially extends over the entire recess 26 or exactly over the entire recess 26. Preferably the elastic component 34 is detachably arranged, more particularly in a positive-fit, frictional or tractional manner, more particularly by means of Velcro closure, zip, press studs etc. to the guide-support structure 1, more particular to two partial sections 22, 23, 24. However, it is also conceivable that the elastic component 34 is firmly, i.e. preferably non-detachably in a destruction-free manner, connected to the guide-support structure 1. Additionally or alternatively it is conceivable that one or more elastic components 34 are arranged on the rear side of the guide-support structure 1 as connecting elements to connecting the guide-support structure 1 to the body part 6a or the body 6.

FIG. 9b shows a further frontal view of the lower section 16.

FIG. 10a is a view of the upper section 16 in a medial alignment. From this figure it can be seen that, for example, the further partial section 23 has a spherical outer contour. This shape results from as preferably precise an analysis as possible of the surface regions 2, 4, 5 shown in FIG. 7, so that as large an area of the slightly deforming surface regions 2 can be used for connection and thus for the transmission of forces, as with increasing area greater forces can be transmitted or with constant force even greater comfort for the living entity can be generated.

FIG. 10b is a further view of the lower section 16 in a medial alignment. The partial sections 22, 23, 24 shown in FIG. 10a and b are preferably of different lengths, different widths and preferably each have a different external contour, wherein it is particularly preferably conceivable that the partial sections 22, 23, 24 correspond to each other in length, contour, direction of extension and/or width in sections or completely at least in sections or completely. The partial sections 23, 23, 24 are thus particularly preferably designable in dependence on the relevant application, the relevant living entity, etc.

FIG. 11 shows an arrangement by means of which a method can preferably be implemented in the sense of the invention. However, it is conceivable for additional or alternative devices, arrangements, means and/or method to be used. The body part 6 is in the region of a recording device 7 which at least at times surrounds the body part 6 in sections and preferably fully. The measurements recorded by the recording device 7 are supplied to a processor device 10, which is at connected to the recording device 7 at least in signalling terms. The processor device 10 determines or calculates the deformations which occur when the body part 6 of the living entity is examined and/or scanned and/or recorded or surveyed in different load conditions.

The results generated by the process device 10 are preferably outputted via a display device 12, preferably in the form of a screen 13. On the screen 14, simultaneously or consecutively, a 3-D model 14 of the body part 6, corresponding with the statements relating to FIG. 7 and/or a model 15 of a guide-support structure 1 produced by way of the 3-D model 14 can be seen.

### REFERENCE LIST

1 Guide-support structure
2 Slightly deforming body surface
4 Strongly deforming body surface
5 Strongly deforming body surface, more particularly inversely to body surface 4
6a First body part
6b Further body part
7 Recoding device
8 Surface region
10 Processor device
12 Display unit
13 Screen
14 3-D model
15 Model of the guide-support structure
16 First section
18 Second section
20 Planar portion
22 First partial section
23 Further partial section
24 Second partial section
25 Connecting means
26 Recess
27 Further recess
28 Joint
30 Lower side connection
32 Upper side connection
34 Elastic component
55 Further zone
56 First axis of rotation
57 Second axis of rotation
58 Tension means
99 Body part
100 Holder means
102 Holder unit
103 Band
104 Fastening means
106 Body surface line
108 Holder medium
110 Connecting element
110a First connecting element
110b Second connecting element
110c Third connecting element
110d Fourth connecting element
110e Fifth connecting element
112 Core
114 Cover
116 Upper layer
118 Lower layer
X Depth
Y Width
X Lenght

1. A holder means for connecting, more particularly for the defined fastening, of at least one guide-support structure to a living entity, at least comprising a holder unit and
a fastening means for the locally stable arrangement of the holder unit on the living entity,
wherein the holder unit is elongated in design and can be arranged in the direction of extension of a body surface line of the living entity to at least in sections overlap the body surface line,
wherein the body surface line is such that between two different body load conditions it essentially experiences no change in length.

2. The holder means according to claim 1,
wherein
several holder units are envisaged and each of these holder units can be arranged in the direction of extension of a body surface line of the living entity to at least partially overlap the relevant body surface line,
wherein the body surface lines are such that between two different body load conditions they essentially experience no change in length.

3. The holder means according to claim 2,
wherein
the holder units are arranged in such a way that the entirety of the holder units forms a net-like or grid-like form.

4. The holder means according to claim 2,
wherein
an elastic holder medium is envisaged, wherein the holder units are connected to the holder mediumor are an integral part of the holder medium.

5. The holder means according to claim 4,
wherein
the fastening means is formed by at least two interacting holder units and/or by the holder medium.

6. The holder means according to claim 4,
wherein
the holder medium is designed as an item of clothing.

7. The holder means according to claim 4,
characterised in that
the holder unit for attaching the guide-support structure overlaps the holder medium on the side facing the guide-support structure and at least one further holder unit is arranged on the side of the holder medium facing the living entity overlapping the holder medium.

8. The holder means according to claim 1,
wherein
the holder means comprises at least one connecting element or a connection area for attaching the guide-support structure wherein the at least one connecting element or the connection area is arranged on the outside of the holder means and overlapping at least one carrier medium in sections.

9. The holder means according to claim 1,
wherein
the surface of at least one holder unit is designed at least in sections to form a frictional and/or positive-fit connection with the guide-support structure.

10. The holder means according to claim 1,
wherein
at least one holder unit is non-elastically designed.

11. A multiple layer arrangement, at least comprising:
a holder means for attaching, more particularly for the defined fastening, of at least one guide-support structure to a living entity, wherein the holder means at least comprises
a holder unit and
a fastening means for the locally stable arrangement of the holder unit to the living entity,
wherein the holder unit is elongated in design and can be arranged in the direction of extension of a body surface line of the living entity to at least in sections overlap the body surface line,
wherein the body surface line is such that between two different body load conditions it essentially experiences no change in length
and
a guide-support structure, more particularly of an orthesis, for the transmission of forces introducible into a first section of the guide-support structure onto a second section of the guide-support structure at a distance from the first section
wherein the first section is designed for at least indirect and at least in sections planar contacting of a surface region of a body part of the living entity,
wherein at least the first section of the guide-support structure overlaps the holder unit at least in sections.

12. The multiple-layer arrangement according to claim 11,
wherein
for contacting on the body part at least the planar portion of the first section is designed in such a way that
for fulfilment of the function of the guide-support structure it can be applied in a defined alignment vis-à-vis the body part on surface areas which on transition from a first load condition into a second load condition exhibit surface deformations which during the same transition are less than surface deformations of further surface areas of the body part
or
for fulfilment of the function of the guide-support structure it can be applied in a defined alignment vis-à-vis the body part on surface areas which on transition from a first load condition into a second load condition exhibit surface deformations which during the same transition are greater than surface deformations of further surface areas of the body part.
